(19) United States
(12) Patent Application Publication
(10) Pub. No.: US 2017/0136013 A1
(43) Pub. Date: May 18, 2017

(54) Method of Treating Hand-Foot Syndrome and Symptoms Associated Therewith

(71) Applicant: Galderma S.A., Lausanne 30 Grey (CH)

(72) Inventor: Philippe Andres, Peymeinade (FR)

(73) Assignee: Galderma S.A., Lausanne 30 Grey (CH)

(21) Appl. No.: 15/319,429

(22) PCT Filed: Jun. 22, 2015
(86) PCT No.: PCT/EP2015/063930

§ 371 Date: Dec. 16, 2016

Related U.S. Application Data
(60) Provisional application No. 62/016,324, filed on Jun. 24, 2014.

(51) Int. Cl. A61K 31/498; A61K 31/131; A61K 31/137; A61K 9/00; A61K 31/4164

(52) U.S. Cl. CPC A61K 31/498; A61K 9/0014; A61K 31/4164; A61K 31/137; A61K 31/131

(57) ABSTRACT

A method of treating hand-foot syndrome (palmar-plantar erythrodysesthesia) and symptoms associated therewith in a patient undergoing or scheduled to undergo chemotherapy is claimed. The method involves topically administering a pharmaceutical composition comprising an effective amount of an alpha adrenergic receptor agonist, pharmaceutically acceptable salt thereof, or combinations thereof; and a pharmaceutically acceptable carrier to the affected areas of skin or to the hands and feet.

### CROSS REFERENCE TO RELATED APPLICATION

This application asserts priority from U.S. Provisional Patent Application Ser. No. 62/016,324, filed Jun. 24, 2014, the contents of which are incorporated herein by reference.

### BACKGROUND OF THE INVENTION

Cancer treatments have improved greatly throughout the past century with the development of various therapies including surgery, radiation, hormone therapy, chemotherapy, and immunotherapy. Chemotherapy remains one of the most common treatments for cancer. New chemotherapy drugs and combination chemotherapy drug regimens are constantly being developed and tested to increase potency and reduce side-effects.

However, the majority of chemotherapy drugs have side effects that affect a patient's quality of life. One such side effect is hand-foot syndrome, i.e., palmar plantar (palmoplantar) erythrodysesthesia, acral erythema, Burgdorf syndrome, chemotherapy-induced acral erythema, palmar plantar (palmoplantar) dysaesthesia, palmar plantar erythema, Plantar palmar erythroderma, toxic erythema, which occurs in 2% of chemotherapy patients, depending upon the type of chemotherapy.

Hand-foot syndrome occurs when chemotherapy drugs affect the growth of skin cells or capillaries (small blood vessels) in the hands and feet, and less commonly the face, genital areas, and areas affected by pressure or friction such as folds of skin and skin under tight clothing. Once the chemotherapy drugs are out of the blood vessels, the drugs can damage the surrounding tissue. The pathogenesis of hand-foot syndrome is unknown. One theory is that the chemotherapy drugs cause local damage by accumulating in the eccrine sweat ducts. Others believe that overexpression of cyclooxygenase 2 in the skin as a result of chemotherapy results in inflammation.

Hand-foot syndrome usually first appears within days of commencing chemotherapy, although it may take several months and a number of chemotherapy cycles for symptoms to appear. The palms are always involved and, less consistently, the soles, fingers, toes, tops of feet, and backs of hands. Uncommon sites include the face, genital areas, and sites affected by pressure of friction, such as folds of skin and skin under tight clothing.

Mild to moderate hand-foot syndrome includes symptoms on the palms of the hands and/or soles of the feet, including blisters, ulcers, sores, swelling, tingling sensations, burning sensations, tenderness, and tightness of skin. More severe symptoms of hand-foot syndrome include severe pain and difficulty walking or using hands, due to the blisters, ulcers, sores, cracked, flaking, or peeling skin. Sometimes the symptoms are so severe that chemotherapy regimens must be altered or stopped until the symptoms subside.

There is a need for new therapies to treat and prevent hand-foot syndrome so patients' symptoms are not so severe as to necessitate altering or stopping chemotherapy regimens.

### BRIEF DESCRIPTION OF THE DRAWINGS

FIG. 1 shows the results of evaluation of the effect of brimonidine tartrate after topical administration on the murine UVB-induced epidermal hyperplasia model and, in particular, the response to one UVB-irradiation of mouse skin and impact of brimonidine tartrate treatment upon epidermis thickness.

FIG. 2 shows the results of evaluation of the effect of brimonidine tartrate after topical administration on the murine UVB-induced epidermal hyperplasia model and, in particular, the response to one UVB-irradiation of mouse skin and impact of brimonidine tartrate treatment upon keratinocyte proliferation.

### SUMMARY OF INVENTION

In one embodiment, the invention relates to a method of treating hand-foot syndrome (palmar-plantar erythrodysesthesia) and symptoms associated therewith in a patient undergoing chemotherapy, including topically administering to the affected areas of skin a pharmaceutical composition including an effective amount of an alpha adrenergic receptor agonist, pharmaceutically acceptable salt thereof, or combinations thereof; and a pharmaceutically acceptable carrier during the course of the chemotherapy.

Preferably, the alpha adrenergic receptor agonist is an alpha-1 adrenergic receptor agonist or an alpha-2 adrenergic receptor agonist. The alpha-1 adrenergic receptor agonist or alpha-2 adrenergic receptor agonist is preferably brimonidine, tetrahydrozaline, naphazoline, xylometazoline, epinephrine, norepinephrine, oxymetazoline, phenylephrine, or methoxyamine. Most preferably, the alpha-2 adrenergic receptor agonist is brimonidine or pharmaceutically acceptable salts thereof.

The affected areas of skin include the hands, feet, face, genitals, or combinations thereof. Areas of the hands and feet specifically affected include the palms of the hands, soles of the feet, fingers, toes, top of the feet, back of the hands, or combinations thereof. The palms of the hands are almost always affected.

The pharmaceutically acceptable carrier is preferably a gel, cream, ointment, lotion, or emulsion.

The brimonidine or pharmaceutically acceptable salt thereof is preferably present in an amount of from about 0.5% by weight to about 5% by weight of the composition. More preferably, the brimonidine or pharmaceutically acceptable salt thereof is present in an amount of from about 0.5% by weight to about 2% by weight of the composition.

In a preferred aspect of the invention, the carrier is a gel and the brimonidine or pharmaceutically acceptable salt thereof is present in an amount of about 0.33% by weight of the gel.

In another embodiment, the invention relates to a method of reducing the symptoms of hand-foot syndrome (palmar-plantar erythrodysesthesia) in a patient scheduled to undergo chemotherapy, the method including topically applying a pharmaceutical composition including an effective amount of an alpha adrenergic receptor agonist, pharmaceutically acceptable salt thereof or combinations thereof; and a pharmaceutically acceptable carrier to the hands and feet of the patient prior to undergoing the chemotherapy.

In a preferred embodiment, the pharmaceutical composition is applied three to four hours before administration of the chemotherapy.

### DETAILED DESCRIPTION

The invention relates to a method of treating hand-foot syndrome, i.e., palmar-plantar erythrodysesthesia, and symptoms associated therewith, involving topical administration of a pharmaceutical composition including an alpha adrenergic receptor agonist, pharmaceutically acceptable salt thereof, or combinations therefore; and a pharmaceutically acceptable carrier to the site of the affected areas of skin before, during, and/or after the course of the chemotherapy.

Symptoms of hand-foot syndrome include blisters, ulcers, sores, swelling, tingling sensations, burning sensations, tenderness, tightness of skin, cracked skin, flaking skin, or peeling skin on various parts of the body. The symptoms occur most commonly on the hands and feet, such as the palms, fingers, back of hands, toes, top of feet, and soles. Less commonly, symptoms can occur on the face, genital areas, and sites affected by pressure or friction such as folds of skin and skin under tight clothing.

Alpha adrenergic receptor agonists are well known in the art. In a preferred embodiment, the alpha adrenergic receptor agonist may be an alpha-1 or alpha-2 adrenergic receptor agonist. The alpha adrenergic receptor agonists included in the invention may or may not show selectivity for either the alpha-1 or alpha-2 adrenergic receptors. For example, some may be considered as being both alpha-1 and alpha-2 adrenergic receptor agonists. More preferably, the alpha adrenergic receptor agonist may be a selective alpha-1 or a selective alpha-2 adrenergic receptor agonist.

Examples of selective alpha-1 adrenergic receptor agonists include oxymetazoline, phenylephrine, and methoxyamine. Examples of selective alpha-2 adrenergic receptor agonists include brimonidine, tetrahydrozaline, naphazoline, xylometazoline, epinephrine, and norepinephrine.

The chemical structures of some selective alpha-1 and selective alpha-2 adrenergic receptor agonists are shown below.

| Chemical Structure | Name |
|---|---|
| 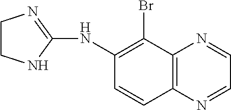 [SMILES: Brc1c(NC2=NCCN2)ccc2nccnc12] | (5-Bromo-quinoxalin- 6-yl)-(4,5-dihydro-1H- imidazol-2-yl)-amine (Bromonidine) |
| 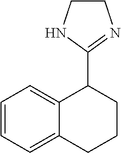 [SMILES: c1ccc2c(c1)CCCC2C1=NCCN1] | Tetrahydrozaline |
| 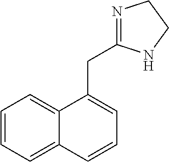 [SMILES: c1ccc2c(CC3=NCCN3)cccc2c1] | Naphazoline |
| 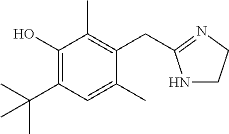 [SMILES: Cc1cc(C(C)(C)C)c(O)c(C)c1CC1=NCCN1] | Oxymetazoline |
| 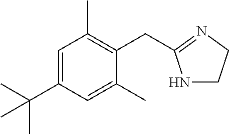 [SMILES: Cc1cc(C(C)(C)C)cc(C)c1CC1=NCCN1] | Xylometazoline |
| 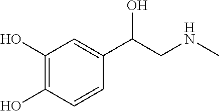 | Epinephrine |
| 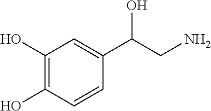 [SMILES: NCC(O)c1ccc(O)c(O)c1] | Norepinephrine |
| 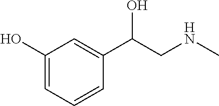 | Phenylephrine |
| 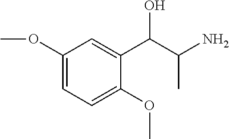 | Methoxyamine |

Brimonidine and its pharmaceutically acceptable salts are preferred embodiments of the invention. Preferably, the active ingredient of the composition is brimonidine tartrate.

The alpha-1 adrenergic receptor agonist, alpha-2 adrenergic receptor agonist, or pharmaceutically acceptable salt thereof may be administered alone or in combination with one or more alpha-1 adrenergic receptor agonist, alpha-2 adrenergic receptor agonist, or pharmaceutically acceptable salt thereof. For example, the active ingredients in the pharmaceutical composition may include brimonidine tartrate and oxymetazoline hydrochloride.

Pharmaceutically acceptable salts for each alpha adrenergic receptor agonists are well known in the art. Pharmaceutically acceptable salt means those salts of compounds of the invention that are safe and effective for topical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. Pharmaceutically acceptable salts are discussed in BERGE ET AL., 66 J. PHARM. SCI. 1-19 (1977), incorporated herein by reference.

Pharmaceutical compositions include any formulations which are pharmaceutically acceptable for topical delivery of the compounds of the invention. The choice of topical formulation will depend on several factors, including the physiochemical characteristics of the particular compound(s) of the invention and of other excipients present, their stability in the formulation, available manufacturing equipment, and cost constraints.

The pharmaceutically acceptable composition is applied locally to the site of the affected skin of the patient in any conventional manner well known in the art. For example, the composition may be a lotion that can be applied to the palms of the hands and the soles of the feet.

The composition can be applied before, during, and/or after chemotherapy for as long as the symptoms of hand-foot syndrome persist.

The amount of alpha adrenergic receptor agonist applied to the skin is any amount that is effective in treating hand-foot syndrome. Generally the minimum amount of an alpha adrenergic receptor agonist in a topical formulation of the invention applied to the affected skin area is about 0.0001 g/cm², preferably about 0.001 g/cm² of skin surface area. The maximum amount of an alpha adrenergic receptor agonist in a topical formulation of the invention applied to the affected skin area is about 0.05 g/cm² to about 0.008 g/cm² of skin surface area. Typically, one to four applications per day are recommended during the term of treatment.

Dosages and dosing frequency will be determined by a trained medical professional depending on the activity of the compound of the invention, the characteristics of the particular topical formulation, and the general physical condition of the person being treated.

For example, the pharmaceutical composition may be applied before, during, and/or after the course of the chemotherapy. An appropriate time before and after the course of chemotherapy may be determined by a trained medical professional. For example, a doctor may prescribe application of the composition three to four hours prior to the administration of the chemotherapy, during the course of the chemotherapy, and in the five days after the administration of the chemotherapy.

In general, each alpha adrenergic receptor agonist or pharmaceutically acceptable salt thereof is present in a formulation of the invention in a minimum amount of from about 0.05 percent, about 0.1 percent, or about 0.15 percent of the total weight of the formulation. Preferably, an alpha adrenergic receptor agonist or pharmaceutically acceptable salt thereof is present in a formulation of the invention in a maximum amount of about 5 percent, about 2 percent, about 1 percent, or about 0.5 percent of the total weight of the formulation.

It is to be understood that the present invention contemplates embodiments in which each minima is combined with each maxima to create all feasible ranges. For example, each alpha adrenergic receptor agonist or pharmaceutically acceptable salt thereof may be present in a composition of the invention in an amount of from about 0.05 percent to about 1 percent based upon the total weight of the composition or, likewise, from about 0.1 percent to about 1 percent based upon the total weight of the composition.

In one embodiment, the compounds of the invention are delivered to the affected area of the skin in a pharmaceutically acceptable topical carrier. As used herein, a pharmaceutically acceptable topical carrier is any pharmaceutically acceptable formulation that can be applied to the skin surface for topical or dermal delivery of a pharmaceutical or medicament. The combination of a pharmaceutically acceptable topical carrier and a compound of the invention is termed a pharmaceutical composition of the invention. Pharmaceutical compositions of the invention are prepared by mixing a compound of the invention with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference. The discussion of pharmaceutical compositions containing alpha adrenergic receptor agonists from U.S. Pat. No. 7,439,241 is incorporated herein by reference.

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The preferred carriers are gels, creams, ointments, lotions, and emulsions.

### EXAMPLES

### Example 1

Gel Composition

| Ingredient | Weight Percent |
|---|---|
| Brimonidine tartrate | 0.33% |
| Oxymetazoline hydrochloride | 0.2% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 2

Cream Composition

| Ingredient | Weight Percent |
|---|---|
| Brimonidine tartrate | 0.5% |
| Oxymetazoline hydrochloride | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 3

Lotion Composition

| Ingredient | Weight Percent |
|---|---|
| Brimonidine tartrate | 0.25% |
| Amerlate P Isopropyl Lanolate | 0.5% |
| Stearic Acid | 3.0% |
| Glyceryl Stearate | 2.0% |
| Methyl Gluceth-20 | 5.0% |
| Triethanolamine | 1.0% |
| Water | 83.45% |
| Polyquaternium-24 and | 3.8% |
| Hyaluronic Acid (BIOCARE | |
| Polymer HA-24) | |
| Germaben IIE | 1.0% |
| TOTAL | 100% |

### Example 4

A lotion containing 0.25 weight % brimonidine tartrate is administered to the palms of the hands and the soles of the feet of a patient undergoing chemotherapy. The patient applies the lotion to his skin twice a day throughout the course of his chemotherapy.

### Example 5

Brimonidine was tested on the below-described murine model and shown to decrease epidermal hyperplasia and keratinocyte proliferation.

### Materials and Methods

### UVB Irradiation

The back skin of the SKH1 mice was exposed to 120 mJ/cm² UVB using the Biospectra system equipped with UVB sunlamps with a maximum emission peak at 312 nm. Irradiations were performed under isoflurane gaseous anesthesia. Vehicle (PEG400/EtOH/PHY (30/20/50) p/p) or brimonidine at 0.2% or 2% was administered by topical route (100 μl) on a 10 cm²-area on the back using a micropipette. The area is located on the upper part of the back to prevent the animals from licking themselves.

Three applications were performed according to two treatment schedules:
pre-treatment: the first application, 1 h prior the UVB irradiation and then twice more at 23 hours apart
post-treatment: the first application just following the UVB irradiation and then twice more at 23 hours apart.

The compound reference, an EGFR inhibitor at 4%, in ETOH/H2O (76/24) was also administrated in pre-treatment.

One hour after the third topical treatment and one hour before euthanasia, mice received an i.p. injection of 5-ethynyl-2′-deoxyuridine (EdU) at 100 mg/kg. After mouse euthanasia (using cervical dislocation) back skin was removed and immediately fixed in formol. The formol-fixed skin samples were embedded in paraffin and then were submitted to immunohistological studies (epidermal thickness measure and EdU detection). For epidermal thickness, two sections per animal (7 μm) were stained in H&E. Skin histology and the epidermis thickness were analyzed using image analysis (mScope 3.9, Aurora mScope) on scanned slide pictures (NanoZoomer C9600-12, Hamamatsu Photonics K.K). For EdU detection, two or three sections (7 μm) for each animal were submitted to EdU staining: briefly, paraffin sections were rehydrated and were then incubated 30 min with Click-iT® reaction cocktail (Click-iT® EdU Imaging Kit with Alexa Fluor® 594 Azide from Life Technologies). Nuclei were also stained with DAPI (5 mg/ml) diluted in Vectashield Hard Set Mounting medium for Fluorescence ref: H-1400 Vector Laboratories). The number of keratinocyte stained with Alexa Fluor® 594 was counted from digital images (NanoZoomer C9600-12, Hamamatsu Photonics K.K) and the epidermis length of each section determined using an in-house dedicated tool.

### Data Analysis

Data were analyzed using unpaired t test for validation of the UV irradiation and one-way ANOVA with Dunnett's multiple comparison test for the analysis of the treatment effect (Prism 6; GraphPad Software Inc., San Diego, Calif., USA). A P-value of <0.05 was regarded as significant.

### Results

### Brimonidine Decreases Epidermal Hyperplasia and Keratinocyte Proliferation

The effect of brimonidine tartrate after topical administration on the murine UVB-induced epidermal hyperplasia model was evaluated.

The histological analysis of H&E-stained skin sections showed that UVB-irradiation (120 mJ/cm²) on mouse skin produces epidermal acanthosis highly visible 48 hours following the irradiation (epidermis thickness increased by +127% in comparison with vehicle-treated mice). This acanthosis was inhibited fully (−96%) by topical pre-treatment with a reference treatment (EGFR inhibitor at 4%) and partially (−23%) by the application of brimonidine tartrate at 2% (one application one hour before UV irradiation and then twice more at 23 hours apart). However, no effect was observed with a lower dose (0.2%).

In order to exclude a UVB-filter effect of brimonidine and support a pharmacological activity, the same experiments were conducted in post-treatment. Similar results were obtained in this case, with a slight decrease of the epidermal thickness at 0.2%, and the largest and significant decrease of the epidermal thickness at 2% brimonidine tartrate (−16% and −32% respectively).

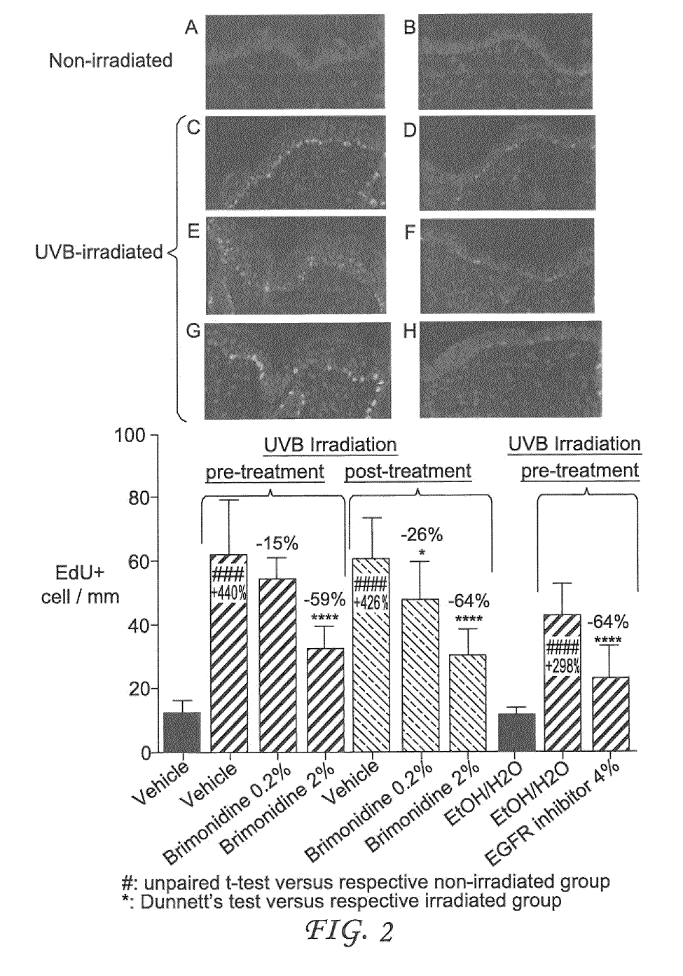

To better characterize the effect of brimonidine tartrate on epidermal hyperplasia, a proliferation marker was analyzed: EdU, a thymidine analogue, was incorporated into cellular DNA during DNA replication and the incorporated EdU was detected through a “click” reaction with a fluorescent Alexa Fluor®594 azide (Zeng, Bain Res. 2010). It was confirmed that one UVB-irradiation produces an increment of proliferating keratinocytes stained with Alexa Fluor®594 (more than 4 fold). As shown in FIG. 2, the reference compound EGFR inhibitor decreased by 64% the number of proliferating keratinocytes. Brimonidine tartrate at 2% in pre- and post-treatment successfully reduced the number of EdU positive cell (−59% and -64% respectively). This effect was also observed with the lower dose, 0.2%, in post-treatment (decrease of 25%).

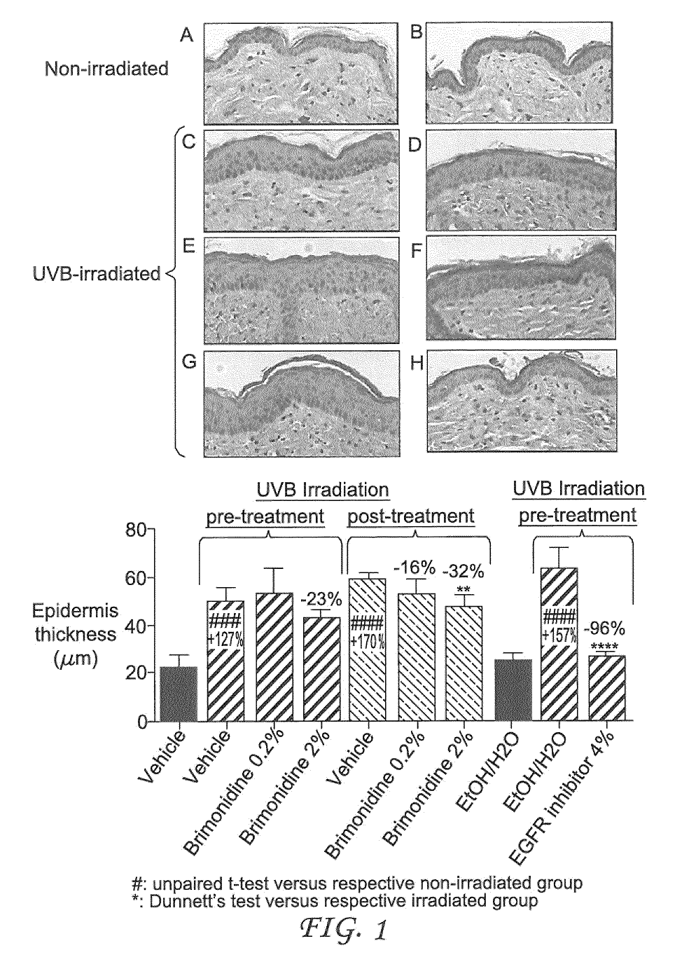

FIG. 1. Response to one UVB-irradiation of mouse skin and impact of brimonidine tartrate treatment upon epidermis thickness.
Vehicle or brimonidine at 2% was administrated by topical route on the back. Top, H&E straining of dorsal skin sections: non irradiated skin treated with the vehicle (A); irradiated skin treated in pre-treatment with the vehicle (C) or brimonidine tartrate 2% (D); irradiated skin treated in post-treatment with the vehicle (E) or brimonidine tartrate 2% (F). The acanthosis was inhibited by topical pre-treatment with an EGFR inhibitor at 4%: non irradiated skin treated with EtOH/H2O (B); irradiated skin treated in pre-treatment with EtOH/H₂O (G) or EGFR inhibitor 4% (H). Bottom, quantification analysis of the effects of brimonidine pre- and post-treatment at 0.2% and 2% on epidermal thickness. The reduction in epidermal thickness caused by a post-treatment with brimonidine tartrate was statistically significant (**p<0.01) for the dose of 2%. EGFR inhibitor was used a reference treatment. Means±SD.
FIG. 2. Response to one UVB-irradiation of mouse skin and impact of brimonidine tartrate treatment upon keratinocyte proliferation.
Vehicle or brimonidine at 2% was administrated by topical route on the back. Top, EdU (pink) and DAPI (blue) staining for proliferating keratinocyte and nuclei respectively: non irradiated skin treated with the vehicle (A); irradiated skin treated in pre-treatment with the vehicle (C) or brimonidine tartrate 2% (D); irradiated skin treated in post-treatment with the vehicle (E) or brimonidine tartrate 2% (F). The acanthosis was inhibited by topical pre-treatment with an EGFR inhibitor at 4%: non irradiated skin treated with EtOH/H₂O (B); irradiated skin treated in pre-treatment with EtOH/H₂O (G) or EGFR inhibitor 4% (H). Bottom, quantification analysis of the effects of brimonidine pre- and post-treatment at 0.2% and 2% on epidermal proliferation. EdU incorporation was calculated as the number of EdU positive cells in the basal layer of the epidermis relative to the epidermis length. The reduction in epidermal proliferation caused by a treatment with brimonidine tartrate was statistically significant (*p<0.05 and ****p<0.0001) for both doses (0.2% and 2%), except the 0.2% dose in pre-treatment. EGFR inhibitor was used as a reference treatment. Means±SD.

1. A method of treating hand-foot syndrome (palmar-plantar erythrodysesthesia) and symptoms associated therewith in a patient undergoing chemotherapy comprising topically administering to the affected areas of skin a pharmaceutical composition comprising an effective amount of an alpha adrenergic receptor agonist, pharmaceutically acceptable salt thereof, or combinations thereof; and a pharmaceutically acceptable carrier during the course of the chemotherapy.

2. A method according to claim 1, wherein the alpha adrenergic receptor agonist is an alpha-1 adrenergic receptor agonist or an alpha-2 adrenergic receptor agonist.

3. A method according to claim 2, wherein the alpha-1 adrenergic receptor agonist or alpha-2 adrenergic receptor agonist is brimonidine, tetrahydrozaline, naphazoline, xylometazoline, epinephrine, norepinephrine, oxymetazoline, phenylephrine, or methoxyamine.

4. A method according to claim 1, wherein the alpha-2 adrenergic receptor agonist, pharmaceutically acceptable salt thereof, or combinations thereof is brimonidine or pharmaceutically acceptable salts thereof.

5. A method according to claim 1, wherein the affected areas of skin are the hands, feet, face, genitals, or combinations thereof.

6. A method according to claim 5, wherein the affected areas of skin are the palms of the hands, soles of the feet, fingers, toes, top of the feet, back of the hands, or combinations thereof.

7. A method according to claim 6, wherein the affected areas of skin are the palms of the hands.

8. A method according to claim 1, wherein symptoms of hand-foot syndrome comprise blisters, calluses, swelling, ulcers, sores, or combinations thereof.

9. A method according to claim 1, wherein the pharmaceutically acceptable carrier is a gel, cream, ointment, lotion, or emulsion.

10. A method according to claim 4, wherein the brimonidine or pharmaceutically acceptable salt thereof is present in an amount of from about 0.5% by weight to about 5% by weight of the composition.

11. A method according to claim 10, wherein the brimonidine or pharmaceutically acceptable salt thereof is present in an amount of from about 0.5% by weight to about 2% by weight of the composition.

12. A method according to claim 11, wherein the carrier is a gel and the brimonidine or pharmaceutically acceptable salt thereof is present in an amount of about 0.33% by weight of the gel.

13. A method of reducing the symptoms of hand-foot syndrome (palmar-plantar erythrodysesthesia) in a patient scheduled to undergo chemotherapy, the method comprising topically applying to the hands and feet of the patient prior to undergoing the chemotherapy a pharmaceutical composition comprising an effective amount of an alpha adrenergic receptor agonist, pharmaceutically acceptable salt thereof, or combinations thereof; and a pharmaceutically acceptable carrier.

14. A method according to claim 13, wherein the alpha adrenergic receptor agonist is an alpha-1 adrenergic receptor agonist or an alpha-2 adrenergic receptor agonist.

15. A method according to claim 14, wherein the alpha-1 adrenergic receptor agonist or alpha-2 adrenergic receptor agonist is brimonidine, tetrahydrozaline, naphazoline, xylometazoline, epinephrine, norepinephrine, oxymetazoline, phenylephrine, or methoxyamine.

16. A method according to claim 13, wherein the alpha-2 adrenergic receptor agonist, pharmaceutically acceptable salt thereof, or combinations thereof is brimonidine or pharmaceutically acceptable salts thereof.

17. A method according to claim 13, wherein the pharmaceutically acceptable carrier is a gel, cream, ointment, lotion, or emulsion.

18. A method according to claim 16, wherein the brimonidine or pharmaceutically acceptable salt thereof is present in an amount of from about 0.5% by weight to about 5% by weight of the composition.

19. A method according to claim 18, wherein the brimonidine or pharmaceutically acceptable salt thereof is present in an amount of from about 0.5% by weight to about 2% by weight of the composition.

20. A method according to claim 19, wherein the carrier is a gel and the brimonidine or pharmaceutically acceptable salt thereof is present in an amount of about 0.33% by weight of the gel.

21. A method according to claim 13, wherein the pharmaceutical composition is applied three to four hours before administration of the chemotherapy.
